(19) United States
(12) Patent Application Publication
(10) Pub. No.: US 2020/0324015 A1
(43) Pub. Date: Oct. 15, 2020

(54) PH-REGULATING WOUND DRESSING

(71) Applicant: PAUL HARTMANN AG, Heidenheim (DE)

(72) Inventors: Markus KETTEL, Heidenheim (DE); Martin JUNGINGER, Hermaringen (DE)

(21) Appl. No.: 16/955,217

(22) PCT Filed: Dec. 14, 2018
(86) PCT No.: PCT/EP2018/084904

§ 371 Date: Jun. 18, 2020

(30) Foreign Application Priority Data
Dec. 21, 2017 (DE) 10 2017 130 893.8

(51) Int. Cl. A61L 15/42; A61L 15/24

(52) U.S. Cl. CPC A61L 15/425; A61L 2400/12; A61L 2300/21; A61L 15/24

(57) ABSTRACT

The present invention relates to wound dressings, more particularly for the need-based treatment of chronic and/or infected wounds, comprising a Bronsted acid and a pH-sensitive polymer, wherein the Bronsted acid is embedded in a matrix of pH-sensitive polymer or is surrounded by pH-sensitive polymer. The present invention also relates to particles containing a Bronsted acid and a pH-sensitive polymer for use in the treatment of wounds, more particularly infected wounds and/or chronic wounds.

The present invention relates to wound dressings, in particular to the need-based treatment of chronic and/or infected wounds.

The healing of skin wounds is based on the ability of skin, epithelium, and connective and supporting tissue to regenerate. The regeneration itself is characterized by a complex process of interconnecting cellular activities that progressively advance the healing process. For example, the literature describes three key phases of healing in a wound, particularly in wounds with loss of tissue. These comprise the inflammatory or exudative phase for hemostasis and wound cleaning (phase 1, inflammatory phase or cleaning phase), the proliferative phase for building up granulation tissue (phase 2, proliferation or granulation phase), and the differentiation phase for epithelialization and cicatrization (phase 3, differentiation or epithelialization phase). It has been found that wound healing is promoted in particular by modern, moist wound treatment. Moist wound treatment employs inter alia wound dressings having a foam layer. Such a foam layer provides regrowing tissue with a matrix that stimulates wound healing and can at the same time absorb and bind relatively large amounts of wound exudate. What has also proven useful is the use of hydrogels to keep wounds moist, which also reduce skin irritation at the wound margin.

It has long been known that wound healing, particularly during the inflammatory and granulation phases of wound healing, can sometimes be impaired. In the context of the present invention, the term wound also includes the wound bed. The wound may contain a wound exudate. Wound healing can be influenced by the wound pH or by the pH of the wound exudate present in the wound. Healthy skin normally has a pH in the acid range, approximately between pH 4.0 and pH 5.7.

In wounds in which wound healing is impaired, particularly in the case of chronic or infected wounds, an alkaline pH can often be observed in the wound or in the wound exudate. Measurement can be carried out for example by in-vitro measurement of the pH of the wound exudate. A shift in pH from acid to alkaline can be caused, for example, by the proliferation of bacteria or by the development of necrosis.

Necrosis and pathological microorganisms can affect the physiological metabolism during the wound healing process. This often leads to local hypoxia and consequent further breakdown of adjoining tissue. The alkaline environment that develops can promote further processes that result in tissue breakdown. In addition, the alkaline environment can stimulate the proliferation of other pathogenic microorganisms and thus additionally hinder wound healing. Chronic wounds in the context of this invention refer to wounds that do not heal within an expected period of 4 to 6 weeks.

The inflammatory phase normally occurs immediately after the injury and lasts about three days. It is characterized by vascular contraction, activation of the coagulation cascade, and complex immunological processes. A fibrin network normally forms, which closes the wound and provides protection from the outside. The release of vasoactive substances (e.g. histamine and serotonin) can trigger a local inflammatory reaction. Adjoining blood vessels may dilate and leukocytes may migrate to the inflammation site as a consequence of increased capillary permeability. These can eliminate microorganisms and tissue necrosis. This allows the wound to be cleaned.

The proliferation or granulation phase that follows on from this usually commences on about the second day after wound formation and can last, for example, up to 14 days. New tissue is established, with vascularization and filling of the defect by granulation tissue. This is the basic prerequisite for subsequent epithelialization. Fibroblasts from the adjoining tissue are able to migrate into the fibrin network and use it as a temporary matrix. The construction of collagen fibers commences. The fibrin scaffold can be broken down by means of fibrinolysis through the enzyme plasmin. The closed vessels can be recanalized.

The maturing of the collagen fibers usually commences with the differentiation or conversion phase, broadly between the sixth and tenth day. The wound contracts through the conversion of fibroblasts into fibrocytes and myofibroblasts. This causes the scar tissue to shrink, resulting in the wound decreasing in size. Inward epithelialization from the wound margin brings wound healing to a close.

Chronic wounds can be defined as wounds in which the healing process deviates from normal wound healing in one or all stages of wound healing. For example, a wound infection can cause acute, normally healing wounds to develop into a chronic wound characterized by a slower rate of healing. The transition from an acute to a chronic wound can occur at any stage of wound healing. Clinically, chronic wounds are defined as wounds that take more than 6-8 weeks to heal, although this definition does not accurately cover all clinical pictures. A chronic wound is more a diagnosis based on the clinical experience of medical personnel.

Chronic wounds develop in particular as a result of mechanical stress (bedsores, pressure ulcers, pressure sores), venous insufficiency (venous leg ulcers, venous ulcers), atherosclerotic vascular changes (arterial leg ulcers, arterial ulcers), neuropathic changes (diabetic foot syndrome, neuropathic ulcers), but also as a consequence of autoimmune diseases, tumors (exulcerating tumors) or radiation damage during tumor therapy.

EP901795 discloses bioabsorbable materials that contain buffer substances and thereby stabilize a pH of 4.5 to 6.5. These materials can also be used as wound-care products, wherein they are laid on a wound and are dissolved by contact with wound exudate. These wound-care products remain on the wound until they are completely absorbed by the body.

WO02/47737 discloses a wound dressing containing an absorbent hydrogel and an additional absorbent layer enclosed by a barrier layer. The barrier layer is more soluble at slightly alkaline pH than in a neutral environment. Any wound exudate is absorbed initially by the hydrogel layer.

WO02/28447 discloses a skin-friendly adhesive that contains a substance that, on contact with pH-neutral water, lowers the pH of the water. The document also discloses a wound dressing coated with said adhesive on the wound-facing side.

EP2726113 discloses a wound-care product containing a foam-like absorbent material that contains buffer substances.

WO2014/027017 discloses the use of buffer substances in wound treatment for the suppression of fibrin formation and also wound-care products that contain buffer substances of this kind.

All of the cited documents disclose wound dressings containing buffer substances for lowering the pH. What all these disclosures have in common is that the buffer substances are released into the wound in an uncontrolled and non-need-based manner.

The disadvantages described in the prior art are overcome by a subject matter of the present invention, as defined in the claims.

The present invention relates to wound dressings comprising a Brønsted acid and a pH-sensitive polymer, in which the Brønsted acid is embedded in a matrix composed of a pH-sensitive polymer or is encased in a pH-sensitive polymer.

In the context of the present invention, a wound dressing is understood as meaning an article that is applied to a wound surface on a patient. A wound dressing according to the invention may be a primary wound dressing, that is to say a wound dressing that is applied directly to the wound being treated, or it may be a secondary wound dressing that is applied to the wound over a wound dressing applied separately therefrom. A suitable wound dressing may comprise a single wound contact layer or it may be composed of several layers. These layers may have different functions.

In a preferred embodiment, a wound dressing according to the invention includes a wound contact layer. In a further preferred embodiment, a wound dressing according to the invention consists of a wound contact layer.

In accordance with the present invention, a material that has no adverse effect on wound healing may be employed as the wound contact layer. A wound contact layer when using the wound dressing of the invention is here in direct contact with the wound. Although the sole purpose of this wound contact layer may be to provide separation between an absorbent layer and the wound being treated, this wound contact layer may also perform further functions as regards the wound dressing and the wound being treated. In particular, a wound dressing according to the invention may include a wound contact layer having a first side and a second side, with the wound contact layer including a hydrogel matrix, a polymer film, a hydrocolloid matrix, a polymer network, a nonwoven, a woven fabric, a knitted fabric, a crocheted fabric or an adhesive.

According to a development of the wound dressing according to the invention, it may be the case that the wound contact layer includes a multiplicity of channels, openings or holes for the passage of liquids. In particular, it is the case that the wound contact layer has channels that provide passage for the wound exudate from the first side to the second side. In this embodiment, it is the case that the first side of the wound contact layer is in direct contact with a wound being treated and the second side of the wound contact layer is in direct contact with the first side of the absorbent layer.

According to a development of the invention, it may also be the case that the wound contact layer has channels, openings or holes that have a diameter of 0.5 to 5 mm. In particular, the wound contact layer has channels, openings or holes that have a diameter of 1 to 3 mm. Very particularly preferably, the wound contact layer has, on the first side that is wound-facing when the wound dressing is applied correctly, openings that have a diameter of 1 to 3 mm, with the second side of the wound contact layer being in direct contact with an absorbent layer.

Further preferably, the wound contact layer may have a multiplicity of channels, openings or holes for the passage of liquids, with the channels, openings or holes on the first side of the wound contact layer occupying an area of not more than 95% of the area of the first side of the wound contact layer. Here it is further preferable that the channels, openings or holes occupy an area of not more than 70%, in particular not more than 50%, in particular not more than 40%, and very particularly preferably not more than 30%, of the area of the first side of the wound contact layer. Very particularly preferably, the wound contact layer has channels, openings or holes that, on the first side of the layer, occupy an area of not less than 5% and not more than 30% of the area of the first side of the wound contact layer. Very particularly preferably, the wound dressing has a wound contact layer that has 2 to 8 holes per cm². It may be the case here that the wound contact layer is a perforated polymer film or a polymer network, in particular a polyurethane film or a polymer network.

According to a further development of the present invention, it may further be the case that a wound dressing according to the invention includes as the wound contact layer a hydrocolloid matrix. This hydrocolloid matrix may consist of an adhesive polymer matrix in which hydrocolloid particles are dispersed. According to the present invention, a hydrocolloid should be understood as meaning a material that is a hydrophilic synthetic or natural polymer material that is soluble or absorbent and/or swells in water. Preferably, a wound contact layer includes a hydrocolloid composed of a synthetic or natural polymer material selected from the group consisting of alginic acid and salts and derivatives thereof, chitin or derivatives thereof, chitosan or derivatives thereof, pectin, cellulose or derivatives thereof such as cellulose ethers or cellulose esters, crosslinked or uncrosslinked carboxyalkyl cellulose or hydroxyalkyl cellulose, polyvinyl alcohol, polyvinylpyrrolidone, agar, guar gum, gelatin or mixtures thereof.

The hydrocolloid may be in the form of fibers or else in the form of particles and/or fibers within a matrix. In particular, the hydrocolloid may be in the form of particles in an adhesive polymer matrix. The adhesive polymer matrix here includes at least one block copolymer selected from the group consisting of AB diblock copolymers and/or ABA triblock copolymers formed from the monomers styrene, butadiene, and isoprene. The proportion of hydrocolloid particles in the wound contact layer may preferably be 10% to 70% by weight based on the total weight of the wound contact layer. A composition of this kind is known for example from EP 1 007 597 B1.

According to a further embodiment of the present invention, it may further be the case that a wound dressing according to the invention includes as the wound contact layer a water-containing hydrogel matrix.

Suitable hydrogel matrices may include gel-forming polymers such as, for example, methyl cellulose, ethyl cellulose, hydroxyethyl cellulose, hydroxypropyl methyl cellulose, carboxymethyl cellulose and the sodium salt thereof, hydroxypropyl cellulose, polyacrylates, polymers of vinyl alcohols, of vinyl esters, of vinyl ethers and of carboxyvinyl monomers, polyvinylpyrrolidone, polyacrylamides, polyethylene glycols, polypropylene glycols, copolymers of polyethylene glycol and polypropylene glycol, polystyrenes polymers of acrylamidomethylpropanesulfonic acid and salts thereof, alginates, hyaluronic acid, and mixtures thereof. Examples of suitable hydrogels are described in published patent applications WO00/45864 and DE102005035879.

In a preferred embodiment, a wound dressing according to the invention includes an absorbent layer. In a further preferred embodiment, a wound dressing according to the invention consists of an absorbent layer.

An absorbent layer is understood as meaning a layer that is able to absorb and store liquid, preferably wound exudate. Preference is given to using plies that have a liquid absorption capacity of at least 1 g/g, more preferably of at least 2 g/g, more preferably of at least 5 g/g, particularly preferably of at least 10 g/g, measured in accordance with DIN-EN 13726-1 (2002).

An absorbent layer may comprise a foam, a mesh tulle, a woven fabric, a knitted fabric, a crocheted fabric, a nonwoven or a fiber material.

Foams are normally understood as meaning materials having cells (open, closed or both) distributed throughout the mass. Such materials thus normally have a bulk density (as defined in DIN EN ISO 845) that is lower than the density of the matrix substance. The density of a foam, preferably a polyurethane foam, may thus be for example between 10 and 110 kg/m³, preferably 15 to 100 kg/m³, more preferably 20 to 95 kg/m³.

In a further embodiment of the invention, further preference is given to using silicone foams, for example those having a density of 50 to 300 kg/m³.

Preference is given to using an open-cell foam as wound dressing (a). A cell is the individual cavity formed during the production of foams, which is partially or completely enclosed by cell walls and/or cell webs. A closed cell is usually a cell that is completely enclosed by its walls and therefore does not communicate with other cells via the gas phase. An open cell is usually a cell that communicates with other cells via the gas phase. In the context of this application, the term open-cell means that the foam contains at least 60% open cells, preferably at least 90% open cells, more preferably at least 98% open cells, in particular essentially 100% open cells, based on the total number of cells. The degree of openness of the foam cells is usually determined in accordance with ASTM D 2856-87, method B).

A suitable foam may have a pore size gradient across the thickness of the foam. The pore size is preferably 1 μm to 1000 μm, more preferably 50 μm to 800 μm. This was determined by microscopy, with microscopic examination of a cross section of a sample; the specified pore size corresponds to the average of 5 randomly selected and measured pores on the cross-sectional surface per sample.

Mesh tulle preferably consists of a hydrophobic material, for example polyester.

Fabrics are generally understood as meaning woven products. These include, for example, cloth, velvet and other textile sheet materials composed of specific thread arrangements, that are essentially perpendicular to one another. The threads in the longitudinal direction are termed warp threads and the threads in the transverse direction are termed weft threads. In order to achieve sufficient fabric strength, the warp and weft threads must be woven closely together and consequently have a closed appearance.

Knitted fabrics are also known as knits or knitwear. Knitted fabrics are classed as knitted goods and are mostly machine-made materials composed of thread systems formed through stitching.

A nonwoven is to be understood as meaning a sheet material or three-dimensional material composed of fibers in a directed or random arrangement that have been mechanically and/or thermally and/or chemically reinforced. Nonwovens differ significantly from woven fabrics, crocheted fabrics, and knitted fabrics.

The nonwoven fabrics of the present invention may comprise fibers of natural or synthetic origin or mixtures thereof. Examples of fibers of natural origin include silk, cellulose, cotton, and wool. Fibers of synthetic origin include synthetic polymers (synthetic fibers) such as viscose, polyacrylates, polyamides, polyimides, polyamide-imides, polyurethanes, polyesters (especially polyethylene terephthalates and polybutylene terephthalates), polyether esters, polyethers, polyacrylonitriles, polyalkenes (especially polyethylenes and polypropylenes), polyurethanes, and polytetrafluorethylenes.

The fiber material is preferably a hydrophilic fiber material. Fibers composed of cellulose, preferably water-insoluble fibers composed of cellulose, in particular largely delignified industrial cellulose fibers, in particular wood pulp fibers, may be used here. Fibers having a fiber length of <5 mm may be used in particular. The fiber material may also comprise hydrophilic fiber material composed of regenerated cellulose, carboxymethyl cellulose, carboxyethyl cellulose, hydroxymethyl cellulose or hydroxyethyl cellulose. The use of a fiber mixture composed of cellulose, regenerated cellulose, carboxymethyl cellulose, carboxyethyl cellulose, hydroxymethyl cellulose or hydroxyethyl cellulose fibers and polyethylene, polypropylene or polyester fibers may also be envisaged.

The absorbent layer may additionally comprise a particle mixture of polyacrylate particles, with the polyacrylate particles containing an interlinked and/or crosslinked polyacrylate.

However, it may also be the case that a transition layer is arranged between the absorbent layer and the wound contact layer. A transition layer of this kind may result from elements of the wound contact layer being mixed with elements of the absorbent layer. This creates a particularly stable connection between the wound contact layer and the absorbent layer. A transition layer may also be in a ply of a further liquid-permeable material layer that is connected both to the wound contact layer and to the absorbent layer. In this case, the transition layer ensures rapid and even transport of any wound exudate from the wound surface to the absorbent layer.

In accordance with an additional, further developed concept of the present invention, the present invention also provides a wound dressing that has a barrier layer between the hydrogel matrix and the hydrophilic polymer foam. A barrier layer of this kind may include, for example, a polymer film provided with openings.

In addition, a wound dressing according to the invention may include a backing layer. This backing layer may consist of different materials. Wound dressings normally have inserted in them textile backing materials, nonwovens, polymer films or polymer foams. This backing layer may have direct contact or indirect contact with the second side of the absorbent layer or with the hydrophilic polymer foam. In the case of direct contact, the backing layer is laminated directly onto the absorbent ply or onto the polyurethane foam, whereas in the case of indirect contact the backing layer is applied to the absorbent layer or the polyurethane foam by means of an adhesive. This adhesive may be applied between the backing layer and the absorbent layer over the entire area or just in sub-regions.

Particular preference is given to using polymer films or polymer foams as the backing layer of a wound dressing according to the invention. Very particular preference is given to polymer films or polymer foams that are impermeable to water and have high permeability to water vapor. Films or foams made from polyurethane, polyether urethane, polyester urethane, polyether-polyamide copolymers, polyacrylate or polymethacrylate are particularly suitable for this purpose. Particularly suitable as the backing layer is a water-impermeable and water-vapor-permeable polyurethane film or a water-impermeable and water-vapor-permeable polyurethane foam. Particular preference as the polymer film is given to a polyurethane film, polyester urethane film or polyether urethane film. Very particular preference is however given to films of this kind having a thickness from 15 to 50 μm, in particular 20 to 40 μm, and very particularly preferably from 25 to 30 μm. The permeability to water vapor of the polymer film of the wound dressing is preferably at least 750 g/m²/24 hours, in particular at least 1000 g/m²/24 hours, and very particularly preferably at least 2000 g/m²/24 hours (measured in accordance with DIN EN 13726). In particularly preferred embodiments, these films have a moisture-proof, adhesive edge section. This edge section ensures that the wound dressing can be applied to and fixed at its intended location. It also ensures that no liquid can escape between the film and the skin surrounding the area to be treated. Particularly preferred adhesives are those that, in a thin application of 20 to 35 g/m², together with the film have a permeability to water vapor of at least 800 g/m²/24 hours and preferably of at least 1000 g/m²/24 hours (measured in accordance with DIN EN 13726).

A Brønsted acid is understood as meaning a substance that, on reacting with suitable solvents, can release hydrogen cations (protons). The products formed from this reaction are a protonated solvent particle and a so-called acid residue, which is the corresponding base, or conjugate base, of the Brønsted acid. Substance mixtures that contain both Brønsted acid and the corresponding base are referred to as buffer substances. The characteristic feature of these mixtures is that, in aqueous solution, they stabilize the pH of the solution, even if amounts of an acid or base are added to the aqueous solution. An acid added to the aqueous solution reacts with the base present in the buffer substance to form a new particle of the Brønsted acid corresponding to the base. No further hydroxonium or hydroxide ions are formed, which means the pH remains virtually unchanged. This pH stabilization remains operational as long as the amount of base particles or acid particles present does not exceed. The quantitative extent of this stabilization is referred to as the buffer capacity.

Wound dressings according to the invention may comprise either a Brønsted acid on its own or a mixture of Brønsted acid and the corresponding base thereof. If a Brønsted acid is used together with the corresponding base thereof, the molar ratio of Brønsted acid and the corresponding base thereof is 100:1 to 1:100, preferably 10:1 to 1:10, more preferably 1:1. An equimolar mixture of Brønsted acid and the corresponding base thereof has the greatest pH stability to the addition of acid or base.

In a preferred embodiment, a wound dressing according to the invention comprises a Brønsted acid and a pH-sensitive polymer, wherein the Brønsted acid is embedded in a matrix composed of a pH-sensitive polymer or is surrounded by pH-sensitive polymer, and additionally a substance that is a Brønsted base corresponding to the Brønsted acid and that is embedded together with the Brønsted acid in a matrix composed of a pH-sensitive polymer or is encased in a pH-sensitive polymer.

Preference is given to using Brønsted acids selected from the group of substances comprising acetic acid, citric acid, lactic acid, glyceric acid, gluconic acid, benzoic acid, aconitic acid, glutaric acid, tartaric acid, phosphoric acid, malic acid, succinic acid, glutamic acid. These Brønsted acids have an advantageous pKa of between 2 and 5, are physiologically safe, and can be used in wound healing.

Particular preference is given to using citric acid, benzoic acid and/or lactic acid as Brønsted acids, optionally together with the corresponding bases thereof. These Brønsted acids have particularly good tissue compatibility.

Wound dressings according to the invention comprise one or more Brønsted acids in one or more different layers or plies. Each of these plies containing a Brønsted acid contains 0.01-100 mmol of Brønsted acid per gram, preferably 0.1-10 mmol of Brønsted acid, more preferably 0.2-5 mmol of Brønsted acid. One or more Brønsted acids, optionally together with the corresponding base thereof, may be present here in a wound contact layer, an absorbent layer, a transition layer, a transport layer, a support layer or a backing layer. It has been found that such a content of Brønsted acid can have a particularly advantageous influence on the pH of the wound exudate.

In the context of the present invention, a pH-sensitive polymer is understood as meaning a substance or a substance mixture that is formed from a multiplicity of repeating monomer units and that in an aqueous solution at a defined temperature and a first pH has a first solubility or rate of dissolution, and at the same temperature and a second pH that is different from the first pH has a second solubility or rate of dissolution that is different from the first solubility or rate of dissolution. The polymer accordingly has different solubilities or rates of dissolution at different pH. Suitable polymers are those that are insoluble in aqueous solution at a pH of less than 6.0 and are soluble in an amount of at least 1% (m/m) at a pH of more than 7.0. Particularly suitable are polymers that at a pH of less than 6.0 in aqueous solution at 20° C. have a rate of dissolution of less than 10 mg/min/g, preferably less than 1 mg/min/g and at a pH of at least 8.0 in aqueous solution at 20° C. have a rate of dissolution of more than 50 mg/min/g, preferably more than 100 mg/min/g. Suitable polymers are cellulose acetate phthalate, cellulose acetate trimellitate, hydroxypropyl methylcellulose acetate succinate, carboxymethyl ethyl cellulose, oxidized regenerated cellulose, polyacrylates, and copolymers and mixtures thereof. Preferred polymers comprise copolymers of methacrylic acid with methyl methacrylate, that is to say a methacrylic acid-methyl methacrylate copolymer, preferably in a ratio of 1:1 to 1:2. Such polymers are available, for example, under the name Eudragit® (Evonik, Darmstadt, Germany). Preferred polymer preparations are available under the names Eudragit® S 100, Eudragit® S 12.5, Eudragit FS 30 D, Eudragit® L 100, Eudragit® L 12.5. Mixtures of these polymer preparations may also be used. These polymers have particularly favorable pH-dependent solution properties. Other suitable polymers are copolymers of methacrylic acid and ethyl acrylate, available for example as Eudragit® L100-55.

The present invention preferably relates to wound dressings which comprise particles that include a Brønsted acid and a pH-sensitive polymer. Particles may be accommodated in different plies of a wound dressing without adverse effect on the physical properties of the respective ply such as absorption capacity, modulus of elasticity or swelling capacity.

In the context of the present application, the term microparticles is understood as meaning all particles, irrespective of the shape thereof, that have an equivalent diameter of the order of 50 nm to 1000 μm. The sub-term microspherules here refers to spherically shaped microparticles, whereas the term microcapsule describes those particles in which a liquid, gaseous or solid core is encased in an outer polymer layer. Particles in which the active substance is present in the polymer in a uniform distribution and in more or less finely divided form are also referred to as micromatrices. The particles used are preferably microparticles, nanoparticles or liposomes.

Microparticles are particles having a particle diameter of between 1 μm and 1000 μm. A distinction is made between microcapsules and microspherules.

Microcapsules comprise a solid or liquid core that is surrounded by a polymer as wall material. Wound-care products produced according to the present invention may comprise microcapsules in which Brønsted acids and optionally the corresponding bases thereof are present in the core in solid and/or liquid and/or dissolved form and are surrounded by a polymer as wall material.

In a preferred embodiment, a wound dressing according to the invention comprises particles, in particular microparticles, that have a core that comprises a Brønsted acid and optionally the corresponding base thereof and is encased in a pH-sensitive polymer.

Microspherules are particles in which a buffer substance is embedded in a polymer matrix without forming a separate capsule wall. In a preferred embodiment, a wound dressing according to the invention comprises particles that include a matrix formed from a pH-sensitive polymer in which a Brønsted acid and optionally the corresponding base thereof are embedded.

Nanoparticles are colloidal solid systems having a diameter of 50-1000 nm. A distinction is made between nanocapsules and nanospherules.

Nanocapsules are solidified micellar systems, solidified microemulsions or encased colloidal solid systems.

Nanospherules are also referred to as nanopellets and are colloidal particles in which a substance is embedded in a polymer matrix.

Liposomes are colloidal spherical lipid vesicles having an aqueous core and a diameter of 20 nm to 3 μm.

Particles having an equivalent diameter of 5 μm to 300 μm are preferably used. Microparticles of this kind are able to penetrate even into fine openings in the material of the ply into which the particles are introduced, and are thus present in a uniform distribution in the respective ply. The small equivalent diameter means that a high surface area is achieved by the totality of the Brønsted acid polymer particles, with the result that any wound exudate immediately comes into close contact with particles containing Brønsted acid, which means that an adequate amount of Brønsted acid can be immediately released if needed.

The equivalent diameter can be determined by laser diffraction, for example using a Malvern Mastersizer 2000. The evaluation is preferably carried out according to the Fraunhofer method.

Suitable particles may be produced in various processes. Those skilled in the art are familiar in principle with numerous processes for producing particles in which a substance is combined with a suitable polymeric material. Reference is, for example, made to the Lehrbuch der Pharmazeutischen Technologie [Textbook of pharmaceutical technology] by the authors Bauer, Frömming, and Führer, which was published in 1999 in its sixth edition by the Wissenschaftliche Verlagsgesellschaft Stuttgart.

Some suitable production processes are outlined below.

In solvent extraction/evaporation processes, droplets of an emulsion of an organic polymer solution, into which a Brønsted acid and optionally the corresponding base thereof can be incorporated in a variety of ways, are introduced into a liquid in which the organic solvent of the disperse phase is soluble and is thereby extracted from the droplets. The Brønsted acid and optionally the corresponding base thereof can either be dissolved together with the polymer in the disperse phase or dispersed therein in a finely pulverized form. Likewise, aqueous solutions of a Brønsted acid and optionally the corresponding base thereof can be emulsified in the disperse phase, as a result of which a double emulsion forms with the continuous, external phase. Droplet formation is achieved with the aid of stirrers or static mixers. This process step largely determines the average particle size and the particle size distribution of the respective end product. In the simplest case, a mixing vessel is charged with the external phase and the acid-containing polymer solution is added at an adequate stirring speed. The reverse process, that is to say the addition of the external phase to the stirred polymer solution, is also possible, in which case droplet formation takes place with phase inversion. Removal of the polymer solvent results in hardening of the droplets that have formed and conversion into microspherules. If the solvent used is sufficiently soluble in the external phase or if the latter is present in sufficiently high excess, solvent removal from the emulsion droplets takes place purely by an extraction process. However, if the distribution equilibrium does not lie sufficiently toward the extractant side, the process must be promoted by continuous removal of the polymer solvent, for example by evaporation or dialysis. In view of the high inclusion efficiency, the extraction liquids used are preferably ones in which the Brønsted acid is insoluble or only sparingly soluble. The rate of solvent removal is a key influencing factor that can be exploited to selectively control the internal structure and hence the release profile of the microparticles. After the extraction/evaporation process, the resulting microparticles are filtered or centrifuged off, washed, and dried under conditions that are mild for the material.

In the phase separation process, the Brønsted acid and optionally the corresponding base thereof in aqueous solution or in the form of a micronized solid substance is emulsified or suspended in an organic polymer solution, for example dichloromethane, ethyl acetate, acetonitrile. The addition of a phase-separation agent, for example silicone oil, results in coacervation of the polymer. Through the adsorption of the coacervate onto the surface of the aqueous droplets or of the solid acid particles, these become covered by an initially still very soft polymer shell. Washing out the phase-separation agent with a solvent in which the polymer does not dissolve, for example hexane or heptane, causes the shell to harden, which then allows the microcapsules formed to be separated.

In the spray-drying process, a joint solution of Brønsted acid, optionally the corresponding base thereof, and polymer in an organic solvent or a suspension of the Brønsted acid and optionally the corresponding base thereof in the organic polymer solution is atomized into a hot stream of air. The dry product is separated from the stream of air by a cyclone. With this process, particles are obtained in which a Brønsted acid and optionally the corresponding base thereof are embedded in a matrix of pH-sensitive polymer. Particles of this kind can be produced particularly inexpensively.

In a further suitable production process, a Brønsted acid and optionally the corresponding base thereof in powder form are placed in a coating pan and sprayed with a suitable polymer solution or polymer dispersion. This results in the formation of microcapsules.

In a further suitable production process, a Brønsted acid and optionally the corresponding base thereof in powder form are sprayed in the fluidized bed with a suitable polymer solution or polymer dispersion. A particularly suitable process is the Wurster process.

In a further suitable process, a buffer substance is added to a solution of a suitable monomer and the monomer is induced to polymerize. This creates particles in which the buffer substance is embedded in a polymer matrix.

Particles used in the context of the present invention may have a mass ratio of Brønsted acid to polymer of between 100:1 and 1:100, preferably between 10:1 and 1:10, more preferably between 10:1 and 5:1. On contact with a wound exudate having an elevated pH, the Brønsted acid contained in such particles is released at a particularly advantageous rate.

The present invention also relates to particles that include a Brønsted acid, optionally the corresponding base thereof and a pH sensitive polymer, for use in the treatment of wounds, preferably infected wounds and/or chronic wounds.

In a preferred embodiment of the present invention, the treatment of wounds, preferably in secondary wound healing, is a phase-appropriate wound treatment. In the context of this application, phase-appropriate wound therapy is understood as meaning that the wound therapy meets the specific needs of the wound in the individual phases.

Thus, phase-appropriate treatment may be selectively carried out in one or more phases of wound healing. (In conventional wound treatments, by way of contrast, the same treatment is carried out throughout all phases.) Preference is thus given to using the particles according to the present invention in the cleaning phase and/or in the granulation phase, since disruption of the pH environment occurs most frequently in these phases.

A wound dressing according to the invention may comprise a Brønsted acid and optionally the corresponding base thereof in a wound contact layer. The wound contact layer may include a liquid-permeable material. Suitable materials are textile materials such as nonwovens, woven fabrics, crocheted fabrics or knitted fabrics, or non-textile materials such as perforated films, plastic nets or the like. This liquid-permeable material may first be impregnated with a solution of a Brønsted acid and optionally the corresponding base thereof and then completely dried, with the result that the Brønsted acid and optionally the corresponding base thereof is completely contained in the appropriate material. The material thus obtained may then be immersed in a solution or dispersion of a suitable pH-sensitive polymer, whereupon the solvent or dispersant evaporates. It is likewise possible to spray the resulting material with a solution or dispersion of a suitable pH-sensitive polymer and then to dry it. In both cases, a uniform coating of the material impregnated with a Brønsted acid and optionally the corresponding base thereof is achieved. To achieve a higher layer thickness, the described coating process may be repeated several times. Preference is given to materials that are coated with a layer of pH-sensitive polymer one to ten times in the described manner. Alternatively, the material may be impregnated or sprayed with an aqueous suspension that contains microparticles of Brønsted acid, optionally the corresponding base thereof, and pH-sensitive polymer. The material is then completely dried again, with the result that the microparticles remain in the material. If the wound contact layer is a semi-solid preparation such as an ointment, cream, paste or gel, preferably a hydrogel, a dispersion of particles including a Brønsted acid, optionally the corresponding base thereof, and a pH-sensitive polymer may be directly incorporated into the preproduced semi-solid preparation, for example by stirring or trituration. A dispersion of particles including a Brønsted acid, optionally the corresponding base thereof, and a pH-sensitive polymer may also be dissolved or dispersed in one of the constituents of the semi-solid preparation and then further processed with the other necessary constituents into the finished semi-solid preparation.

Such a wound contact layer has the advantage that any wound exudate immediately comes into contact with Brønsted acid contained in the wound contact layer, with the result that an unfavorably alkaline pH in the wound exudate can be corrected immediately.

Such a wound contact layer may be used on its own as the primary wound dressing, together with secondary wound dressings applied thereon, or as a layer in a multilayer wound dressing.

A wound dressing according to the invention may comprise an absorbent layer. This absorbent layer may first be impregnated with a solution of a Brønsted acid and optionally the corresponding base thereof and then completely dried, with the result that the Brønsted acid and optionally the corresponding base thereof is completely contained in the appropriate material. The material thus obtained may then be immersed in a solution or dispersion of a suitable pH-sensitive polymer, whereupon the solvent or dispersant evaporates. It is likewise possible to spray the resulting material with a solution or dispersion of a suitable pH-sensitive polymer and then to dry it. In both cases, a uniform coating of the material impregnated with a Brønsted acid and optionally the corresponding base thereof is achieved. To achieve a higher layer thickness, the described coating process may be repeated several times. Preference is given to materials that are coated with a layer of pH-sensitive polymer one to ten times in the described manner.

Alternatively, the material may be impregnated or sprayed with an aqueous suspension that comprises microparticles of Brønsted acid, optionally the corresponding base thereof, and pH-sensitive polymer. The material is then completely dried again, with the result that the microparticles remain in the material.

Such an absorbent layer may comprise large amounts of Brønsted acid, with the result that it is able to correct even a particularly high pH in a wound exudate or the pH of large amounts of unfavorably alkaline wound exudate.

Such an absorbent layer may be used on its own as the primary wound dressing, together with secondary wound dressings applied thereon, or as a layer in a multilayer wound dressing.

### EXAMPLES

### Example 1: Encased Wound Contact Layer

A polyamide knitted fabric measuring 10 cm×10 cm and having a basis weight of 85 g/m² is impregnated with 12.75 mL of a 0.3 mM solution of lactic acid. After drying completely, all the lactic acid remains in the polyamide knitted fabric. The resulting material is impregnated with a commercially available organic solution of Eudragit® S 12.5 by immersing it in the solution for 10 s. The material thus impregnated is dried. After drying completely, a wound contact layer completely coated with pH-sensitive polymer remains. To obtain a sufficient layer thickness, the coating process is repeated five times.

### Example 2: Encased Absorbent Layer

To achieve pH stabilization in the pH range from pH 4 to pH 4.5 and to achieve a buffer capacity of about 0.3-0.4 mmol NaOH, a polyurethane foam (Vivo MCF03, AMS) was treated per gram of foam material with 15 ml of an aqueous solution containing benzoic acid and sodium benzoate, each in a concentration of 0.04 mol/L. After impregnation, the polyurethane foam was dried completely. The product thus obtained was impregnated with a commercially available organic solution of the pH-sensitive polymer Eudragit S 12.5 (methacrylic acid-methyl methacrylate 1:2 copolymer). After drying completely, a foam coated with polyacrylate remains.

To obtain a sufficient layer thickness, the coating process is repeated one more time.

### Example 3: Production of Microparticles by Spray Drying

Various lactic acid microparticles having polymer contents of 10% to 40% were produced. This was done by producing in each case 300 ml of a dispersion, which was then spray dried using a Büchi 190 Mini Spray Dryer. The compositions of the dispersions are listed in the following table:

| | | | Eudragit | |
|---|---|---|---|---|
| | | | FS 30 D | |
| | | Lactic | (polymer | |
| Number | % Polymer | acid | present) | Water |
| MP 01 | 10 | 54.0 g | 20.0 g | 226.0 g |
| | | | (6.0 g) | |
| MP 02 | 20 | 48.0 g | 40.0 g | 212.0 g |
| | | | (12.0 g) | |
| MP 03 | 30 | 42.0 g | 60.0 g | 198.0 g |
| | | | (18.0 g) | |
| MP 04 | 40 | 36.0 g | 80.0 g | 184.0 g |
| | | | (24.0 g) | |

Spray drying was carried out using a Büchi 190 Mini Spray Dryer (Büchi Laboratoriums-Technik, Eislingen, Germany). A drying temperature of 95° C. was chosen. The pump delivery rate with which the dispersion was sprayed was approx. 5 ml/min. A setting of 50% was chosen as the aspirator output. The outlet air temperature was 48° C.

For further processing, the residue obtained was either suspended in water to obtain a suspension or, after renewed trituration, incorporated directly into a suitable medium in a mortar.

### Example 4: Microparticles in a Wound Contact Layer

A polyamide knitted fabric measuring 10 cm×10 cm and having a basis weight of 85 g/m² is impregnated with 1.418 g of a 30% dispersion of lactic acid polymer microparticles obtained according to example 3 and having a polymer content of 10% (MP01). After drying completely, the lactic acid polymer particles remain in the polyamide knitted fabric.

### Example 5: Microparticles in an Absorbent Layer

A polyurethane foam (Vivo MCF03, AMS) was impregnated per gram of foam material with 7.5 g of a 10% aqueous dispersion of lactic acid polymer particles obtained according to example 3 and having a polymer content of 40% (MP04). The foam material thus obtained may be used in a damp or completely dried state.

### Example 6: Microparticles in Hydrogel

A suspension is produced from 30.0 g of hydroxyethyl cellulose, 225.2 g of lactic acid polymer particles produced according to example 3 and having a content of 20% (MP02), and 200.0 g of glycerol. In a second step, 7.0 g of Carbopol 980 NF (polyacrylate, from Lubrizol, Wickliffe, USA) is mixed with 688.0 g of Ringer's solution and stirred for two hours at room temperature. The pH is adjusted to 6.0 through the addition of approx. 75 g of sodium hydroxide solution with a concentration of 1 mol/L and stirring is continued for a further two hours. To this solution is then added very slowly, with uniform stirring, the suspension of hydroxyethyl cellulose, lactic acid polymer particles, and glycerol prepared in the first step. After the addition, stirring is continued for a further two hours. Any air incorporated can subsequently be removed by stirring under reduced pressure. A weakly acidic hydrogel is obtained that contains 2.0 mmol of lactic acid in microencapsulated form per gram. This hydrogel may be applied to a wound directly as an amorphous gel or used as a wound contact layer on an absorbent layer in a multi-ply wound dressing.

1. A wound dressing comprising a Brønsted acid and a pH-sensitive polymer, wherein the Brønsted acid is embedded in a matrix composed of a pH-sensitive polymer or is encased in a pH-sensitive polymer.

2. The wound dressing as claimed in claim 1, wherein the Brønsted acid is contained in particles that include the Brønsted acid and the pH-sensitive polymer.

3. The wound dressing as claimed in of claim 2, wherein the particles have a diameter from 50 nm to 1000 μm.

4. The wound dressing of claim 2, wherein the particles have a core that contains the Brønsted acid and that is encased in the pH-sensitive polymer.

5. The wound dressing of claim 2, wherein the particles comprise a matrix composed of the pH-sensitive polymer, in which the Brønsted acid is embedded.

6. The wound dressing of claim 1, wherein the wound dressing additionally contains a substance that is a Brønsted base corresponding to the said Brønsted acid and that is embedded together with the Brønsted acid in a matrix composed of pH-sensitive polymer or is encased in a pH-sensitive polymer.

7. The wound dressing as claimed in of claim 1 wherein the wound dressing is a wound contact layer.

8. The wound dressing as claimed in of claim 1 wherein the wound dressing is an absorbent layer.

9. The wound dressing of claim 8 wherein the Brønsted acid is contained in the absorbent layer.

10. The wound dressing of claim 7, wherein the Brønsted acid is contained in the wound contact layer.

11. The wound dressing of claim 10, wherein the Brønsted acid is contained in an amount of 0.01-100 mmol per gram in the wound contact layer which contains the Brønsted acid.

12. The wound dressing of claim 1, wherein the Brønsted acid is a substance selected from the group comprising acetic acid, citric acid, lactic acid, glyceric acid, gluconic acid, benzoic acid, aconitic acid, glutaric acid, tartaric acid, phosphoric acid, malic acid, succinic acid, glutamic acid, and combinations thereof.

13. The wound dressing of claim 1, wherein the pH-sensitive polymer is a methacrylic acid-methyl methacrylate copolymer.

14. A method of treating wounds comprising employing a particle comprising a Brønsted acid and a pH-sensitive polymer.

15. The method of claim 14, wherein the wounds are infected wounds.

16. The method of claim 15, wherein the wounds are chronic wounds.

17. The wound dressing of claim 2, wherein the particles have a diameter from 5 μm to 300 μm.

18. The wound dressing of claim 1, wherein the wound dressing includes a wound contact layer.

19. The wound dressing of claim 1, wherein the wound dressing includes an absorbent layer.

20. The wound dressing of claim 9, wherein the Brønsted acid is contained in an amount of 0.01-100 mmol per gram in the absorbent layer which contains the Brønsted acid.
